(19) United States
(12) Patent Application Publication
(10) Pub. No.: US 2021/0112768 A1
(43) Pub. Date: Apr. 22, 2021

(54) Methods and Compositions for Nutrient Enrichment in Plants

(71) Applicant: X Development LLC, Mountain View, CA (US)

(72) Inventor: Anupama Thubagere Jagadeesh, San Jose, CA (US)

(21) Appl. No.: 16/802,545

(22) Filed: Feb. 26, 2020

Related U.S. Application Data
(60) Provisional application No. 62/810,648, filed on Feb. 26, 2019.

(51) Int. Cl. A01H 17/00; A01G 18/10

(52) U.S. Cl. CPC A01H 17/00; A01G 18/10

(57) ABSTRACT

This disclosure describes compositions and methods for delivering nutrients (e.g., nitrogen) to plants.

### CROSS-REFERENCE TO RELATED APPLICATIONS

This application claims priority under 35 U.S.C. § 119(e) to U.S. Application No. 62/810,648 filed Feb. 26, 2019. This application is incorporated by reference in its entirety.

### TECHNICAL FIELD

This invention relates to methods and compositions of nutrient enrichment in plants.

### BACKGROUND

The environmental impact of using nitrogen fertilizer can be significant. In some instances, up to 30% washout of nitrogen from soil-applied fertilizer has been observed. The use of nitrogen fertilizer is usually an inefficient use of resources and can lead, for example, to more expensive food production, pollution of groundwater, depletion of other soil nutrients, and an increase in “dead” zones. Alternate, more efficient ways of delivering nitrogen to plants are desirable.

### SUMMARY

Compositions and methods for delivering nitrogen to plants are described herein. The compositions and methods described herein do not require the use of genetically modified organisms (GMOs) and can significantly improve the nutritional quality available to each plant.

For example, methods of selecting for symbiotic fungi and companion microbe nodules are provided, and methods of using such symbiotic combinations to provide nitrogen directly to plants and plant tissues are provided. As described herein, a symbiotic fungi and companion microbe (e.g., bacteria) are able to fix biological nitrogen and transfer the fixed nitrogen to the plant. In addition, a symbiotic fungi and companion microbe can enhance phosphorus and potassium, and other micronutrient uptake, increase root volume, increase water retention so as to improve drought resistance. Ultimately, a symbiotic fungi and companion microbe can result in a 20% increase in plant yield.

As exemplified below, the methods described herein typically include culturing the appropriate fungal species and companion microbial species and integrating the microbe into the fungus and plant system, after which the bacteria fix nitrogen and transfer the nitrogen to the fungus and, ultimately, the plant.

In one aspect, methods of delivering nutrients to a plant are provided. Such methods typically include contacting a plurality of plant seeds with selected bacteria and a selected mycorrhizal fungus; planting the plant seeds; and allowing plants to grow from the plant seeds, wherein precursors produced by the bacteria are provided to the plants via the fungus, and the plants utilize the precursors.

In some embodiments, the plant seeds include C₃ plant seeds, C₄ plant seeds, or both. In some embodiments, the plant seeds are from a cereal plant.

In some embodiments, the selected bacteria includes a single strain. In some embodiments, the selected bacteria includes a plurality of strains. Representative selected bacteria include Azospirillum brasilense, Azospirillum lipoferum, Azotobacter, Burkholderia Unamae, Gluconacetobacter diazotrophicus, Herbaspirillum seropedicae, Paenibacillus brasilensis, or Paenibacillus durus.

In some embodiments, the selected mycorrhizal fungi is Glomus intraradices or Rhizophagus irregularis.

In some embodiments, the method further includes transfecting the fungus with the bacteria. Such a method may further include contacting a peptide with the bacteria to facilitate the transfecting.

In some embodiments, contacting the plurality of plant seeds with the selected bacteria and the selected mycorrhizal fungus includes coating each seed in the multiplicity of seeds with a composition comprising the selected bacteria to yield coated seeds, and contacting the coated seeds with soil comprising the selected mycorrhizal fungus. In some embodiments, coating each seed occurs before or after germination of the seed.

In some embodiments, contacting the plurality of plant seeds with the selected bacteria and the selected mycorrhizal fungus includes planting the plurality of seeds in soil, and providing spores of the selected mycorrhizal fungus to the soil, wherein the spores comprise the selected bacteria.

In some embodiments, contacting the plurality of plant seeds with the selected bacteria and the selected mycorrhizal fungus includes injecting the selected bacteria and the selected mycorrhizal fungus into soil containing or configured to contain the plurality of plant seeds.

In another aspect, modified seeds are provided. Such seeds typically include a plant seed coated in a selected bacterial strain, wherein a plant grown from the coated seed, when germinated and/or grown in the presence of a selected mycorrhizal fungus, is enriched in nutrients compared to a plant grown from a seed not coated with the selected bacterial strain and not germinated and/or grown in the presence of the selected mycorrhizal fungus.

In still another aspect, modified soil mixtures are provided. Such mixtures typically include soil provided with a selected bacteria-fungus mixture comprising a selected bacteria strain and a selected mycorrhizal fungus, wherein the bacteria-fungus mixture causes a plant grown in the modified soil mixture to synthesize compounds from precursors produced by the bacteria strain that are provided to the plant by the selected mycorrhizal fungus.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DETAILED DESCRIPTION OF DRAWINGS

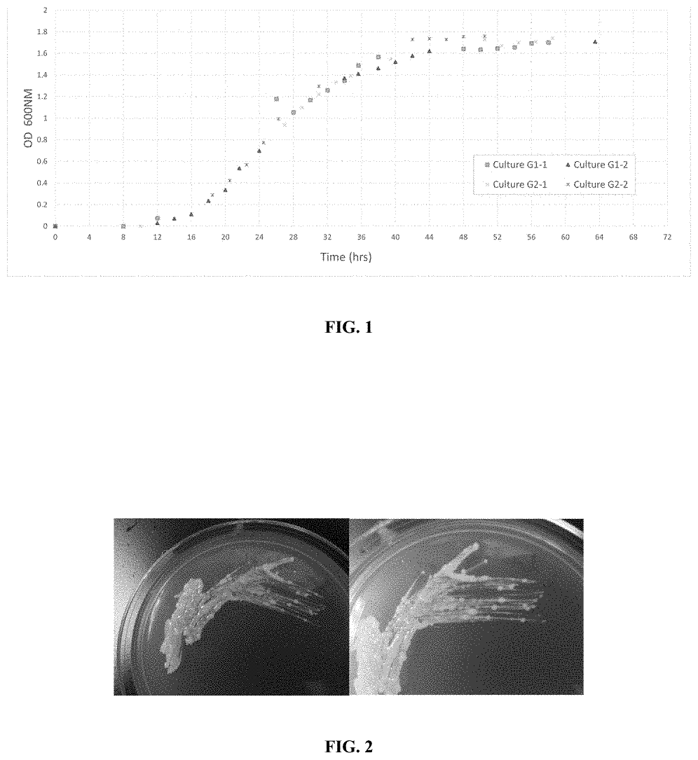

FIG. 1 is a graph showing a growth curve of bacteria.

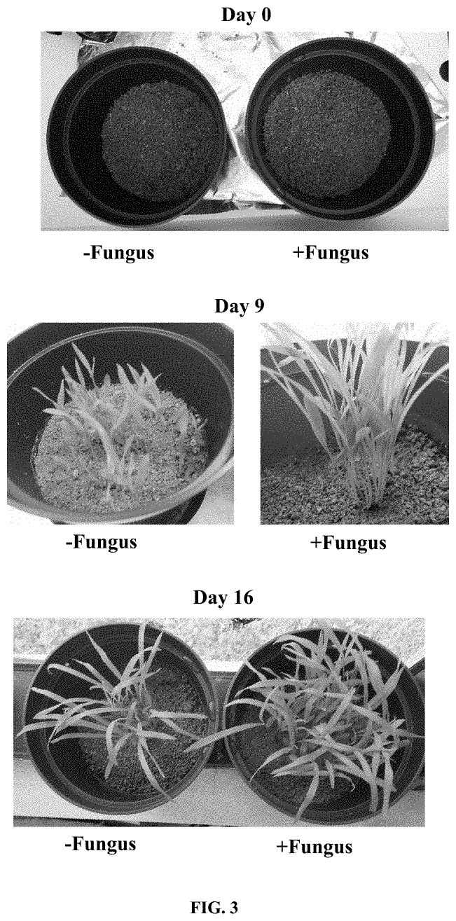

FIG. 2 are photographs showing the morphology of G. diazotrophicus bacteria. The image on the right is an enlarged view of the image on the left.

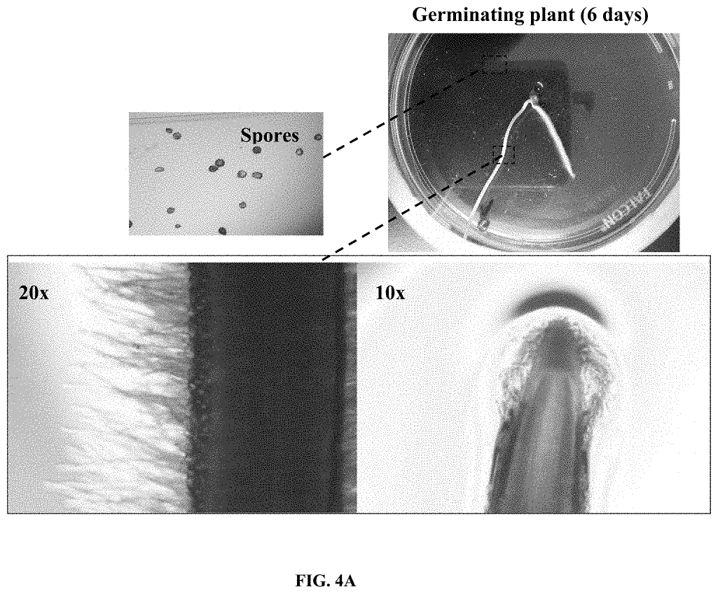

FIG. 3 are photographs that show the effect of the fungus (“+Fungus”) vs. no fungus (“−Fungus”) on plants from day 0 (top), day 9 (middle), and day 16 (bottom).

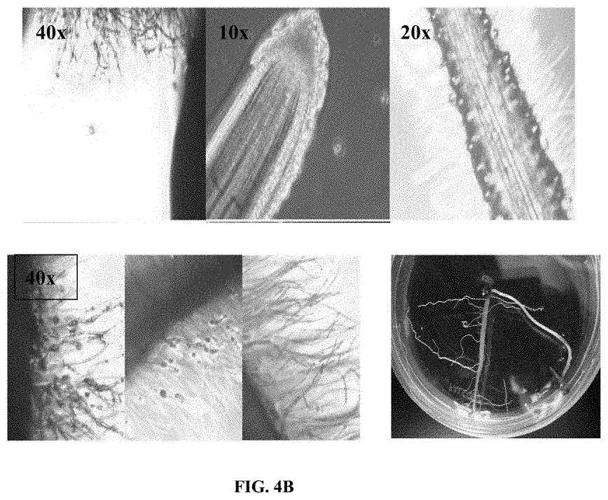

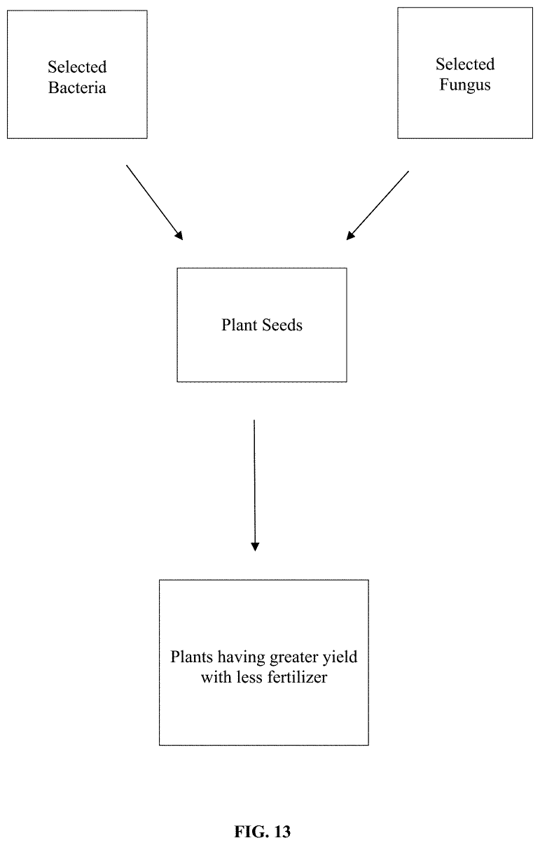

FIGS. 4A-4B are photographs of the roots 6 days (FIG. 4A) or 13 days (FIG. 4B), under the indicated magnification, after inoculation with the fungal spores.

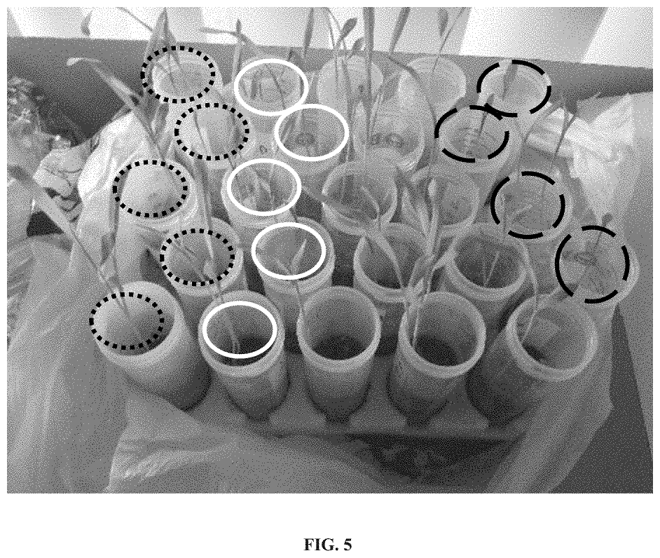

FIG. 5 shows sorghum seeds planted in soil with no additive (control (black, large dashed-line circles on right side of photograph)), fungus only (black, small dotted-line circles on left side of photograph), bacteria only (white, solid circles), or combinations of fungus and bacteria (all others).

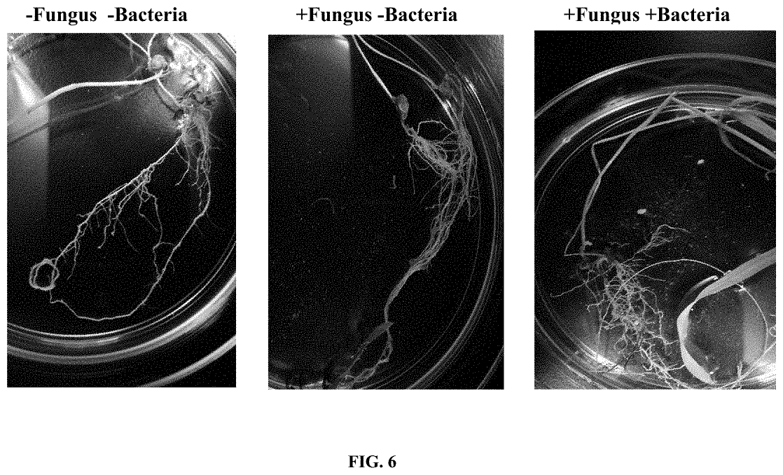

FIG. 6 are photographs showing root morphology after 15 days in the presence (“+”) and absence (“−”) of the fungi and bacteria as indicated.

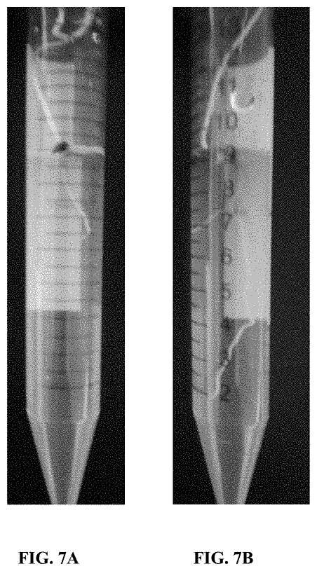

FIGS. 7A-7B are photographs showing root growth in culture in the presence (FIG. 7B) or absence (FIG. 7A) of the fungus.

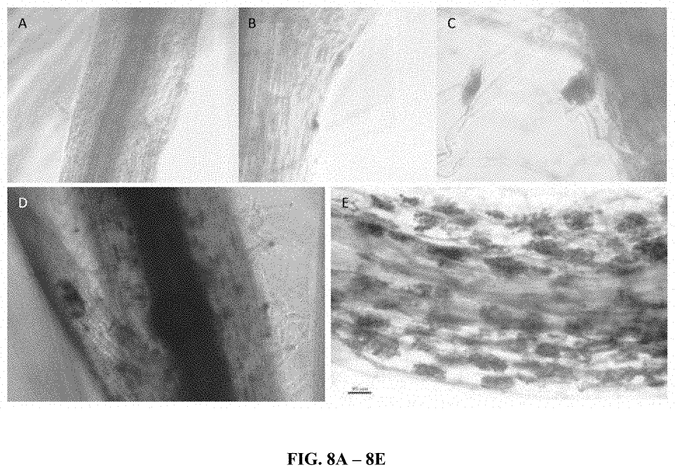

FIGS. 8A-8E show that, microscopically, bacteria could be detected within several different samples of root tissue.

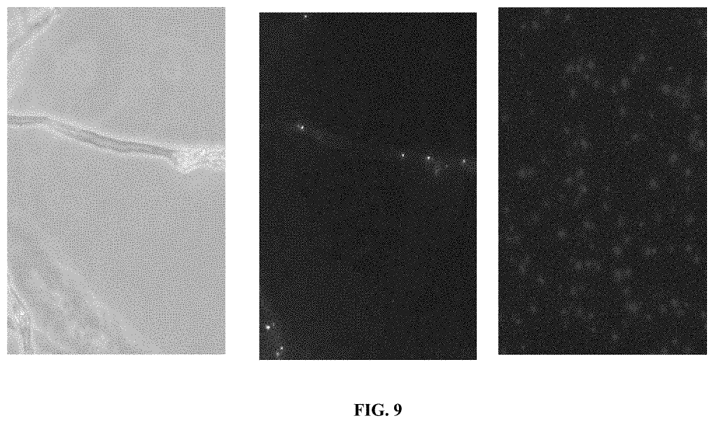

FIG. 9 shows imaging of mixed inoculant sorghum roots using fluorescence microscopy. Sorghum root inoculated with Intraradices fungus and Gluconacetobacter bacterial culture (left); Gluconacetobacter and Burkholderia bacterial culture (middle); or Azotobacter bacterial culture (right).

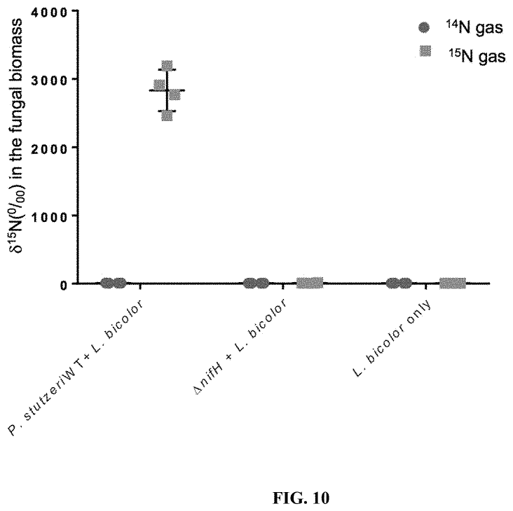

FIG. 10 is a graph of showing the levels of N15 (squares) or N14 (circles) in the fungal biomass in the presence of nitrogen fixing bacteria.

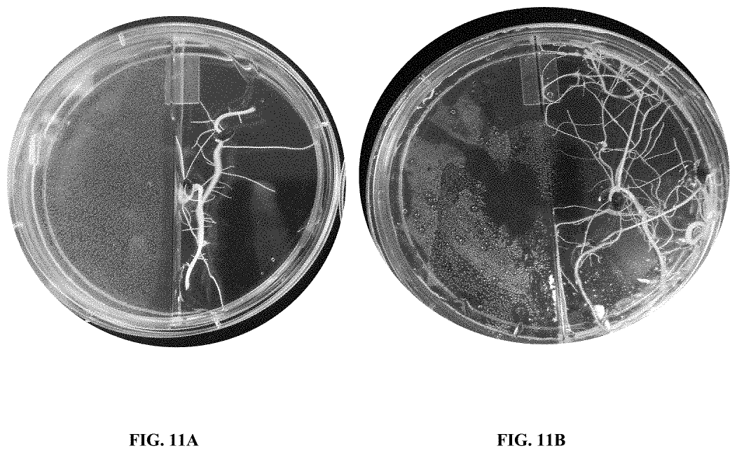

FIGS. 11A-11B are photographs of culture plates showing that nutrient transfer was observed between bacteria and fungi even when the root and the fungi were separated from the bacteria.

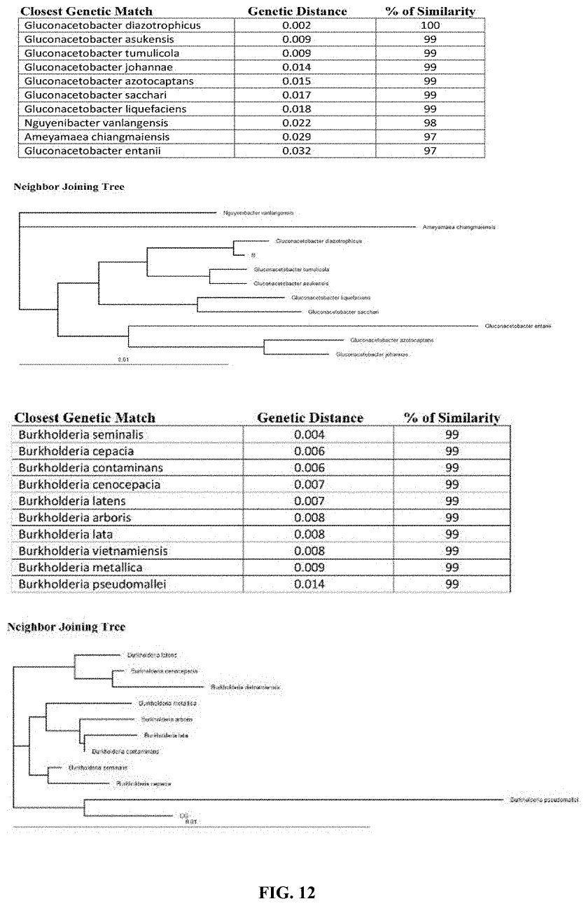

FIG. 12 are the results of genomic sequencing performed on the microorganisms collected from roots. Comparison of the bacterial strains present before coating of the seeds (top) and after growth of the plants (bottom).

FIG. 13 is a flow chart showing one embodiment of the methods described herein.

### DETAILED DESCRIPTION

A non-transgenic approach to modifying the phenotype of a plant without modifying its genotype is described, in which precursors produced by bacteria are provided to plants via existing biological associations with fungi to enhance the production of specific compounds by the plants. Various combinations of fungi, plants, and bacteria can be used. In some implementations, different combinations of bacteria are used to produce precursors that can then be converted into more complex compounds using plants as bioreactors. The combinations of bacteria can be non-transgenically or transgenically engineered to provide the precursors. In some implementations, the bacteria are encapsulated within the fungi (e.g., the fungi have been transformed with the bacteria).

Suitable plants include plants that use C₃ and C₄ carbon fixation pathways (“C₃ plants” and “C₄ plants,”) respectively. Examples of C₃ plants include rice, wheat, soybeans, and trees. Examples of C₄ plants include corn, sugarcane, amaranth, sorghum, millets, and switchgrass. Another class of plants for which the method described herein can be useful are cereals (e.g., maize, rice, wheat, barley, sorghum, millet, oats, rye, triticale, or fonio).

In one example, nitrogen-fixing bacteria are used in conjunction with mycorrhizal fungi to convert atmospheric nitrogen into ammonia that can be used by the plant. The bacteria fix nitrogen, and the fungi serve as a conduit to transfer the fixed nitrogen from the bacteria to the plant. Suitable combinations of bacteria and fungi are selected to transport nutrients into the plants through the roots.

In some implementations, bacteria other than nitrogen-fixing bacteria are used to produce other molecules to be transferred to the fungus. In some implementations, rather than a single strain of bacteria, a microbiome may be introduced into the fungus. The microbiome may be multiplexed to produce variations of molecules that can modify the phenotype of the fungus and hence the phenotype of the plant it associates with. In some implementations, external signaling mechanisms may be used to engulf microbes into high order organisms.

Association of selected seeds with suitable bacteria can be achieved by contacting the seeds with a bacterial culture containing the selected bacteria and germinating the seeds in soil containing selected fungi. In some implementations, contacting the seeds with a bacterial culture includes at least partially coating the seeds with the bacterial culture. The seeds may be contacted before or after germination. Another implementation includes growing a mixture of fungi and bacteria, and introducing the resulting spores into soil containing the seeds before, during, or after watering. Another implementation includes injecting the fungi and bacteria into the soil before or after seeds are planted, yielding a modified soil mixture suitable to provide a growing medium for providing precursors produced by bacteria to plants via existing biological associations with fungi to enhance the production of specific compounds by the plants. Yet another implementation includes initially growing the selected fungi and the plant independently in the soil, such that the fungi contact the roots during growth of the plant.

In some implementations, a peptide (e.g., ralsolamycin, produced by a Ralstonia solanacearum non-ribosomal peptide synthetase-polyketide synthase hybrid) can be used to promote transfection of the fungus with the bacteria. Transfection of the fungus with the bacteria allows the bacteria to reside in the cytoplasm of the fungus, thereby reducing or eliminating leakage of the fixed ammonia or other compounds produced by the bacteria into the soil, and ensuring that most or all of the fixed ammonia or other compounds are transferred to the fungus and then to the plant in a three-way symbiotic method.

The introduction of peptides along with the capability of combining nitrogen-fixing bacteria with the fungus to transport ammonia or other compounds to the plant may enhance production of selected compounds by providing specific precursors to the plant and using the plant as a bioreactor. This ability may be especially advantageous for nutraceuticals. In one example, methods described herein can be used to increase production of certain amino acids by plants. In another example, the production of cannabidiol oil may be increased without producing tetrahydrocannabinol.

Machine learning algorithms (e.g., regression algorithms, instance-based algorithms, regularization algorithms, decision tree algorithms, Bayesian algorithms, clustering algorithms, association rule learning algorithms, artificial neural network algorithms, deep learning algorithms, dimensionality reduction algorithms, ensemble algorithms, or combinations thereof) can be used to design and optimize the groups of microbes suitable to produce precursor molecules to be transferred to the plant via the fungus. The machine learning algorithms may be trained using, among other things, data obtained from experiments using various combinations of the bacterial strains and fungi described herein. Machine learning and bioinformatic tools can also be used to increase the throughput of experiments.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### EXAMPLES

### Example 1

### Experimental Results

As described herein, Step 1 typically includes culturing the appropriate bacterial species. FIG. 1 shows a growth curve of several different bacteria. Liquid cultures were started (t=0) from subcultures grown to OD 0.3-0.4 and seeded 1% (vol/vol) into fresh growth medium. Absorbance (OD600) readings were taken at 2 hour time intervals and data were normalized relative to a reference sample (t=0).

To examine the morphology of the bacteria (G. diazotrophicus), dilutions of liquid cultures were grown and incubated at 30° C. for 48 hrs. Colonies were shiny, circular, cream-white and moderately dry once visible. See FIG. 2.

Also as described herein, Step 2 typically includes culturing the appropriate fungal species. FIG. 3 shows the sporulating fungus grown from day 0-day 16. Sterile soil (700 mL) was inoculated 1:10 with F1 or F2 in 15 cm pots following the protocol for trap cultures (Gopal et al., 2016, Korean J. Soil Sci. Fertiliz., 49:608-13). Pots were seeded with sweet sorghum (˜20 seeds) and left to sprout in direct sunlight for 14 hours/day.

FIG. 4A are photographs of the roots 6 days after inoculation with the fungal spores, and FIG. 4B are photographs of the roots 13 days after inoculation. The fungus colonizes the plant root and grows along with the root. More fibrous divisions indicate fungal extensions farther into the soil, allowing the root to obtain more minerals, water and vitamins from the soil, thereby increasing the growth rate compared to a plant without the fungal inoculation.

Step 3 as described herein typically includes integrating the bacteria with the fungus and the plant root. Sorghum seeds were coated with bacterial culture that included a nitrogen-fixing bacteria (e.g., Azospirillum brasilense, Azospirillum lipoferum, Azotobacter, Burkholderia Unamae, Gluconacetobacter diazotrophicus, Herbaspirillum seropedicae, Paenibacillus brasilensis, Paenibacillus durus). Abuscular mycorrhizal fungi (e.g., Glomus intraradices, Rhizophagus irregularis) were combined with a soil mixture free of added fertilizers. The coated sorghum seeds were planted in the soil mixture and allowed to germinate and grow. As the roots developed, the fungus adhered to the roots and grew along with the plant. FIG. 5 shows that the combined presence of the nitrogen-fixing bacteria and the fungus resulted in a faster growth rate of these experimental sorghum plants than that of control sorghum plants grown from untreated seeds in soil mixture with no added fungus.

After 15 days of growth, association of the fungus and bacteria with the plants was verified by visual inspection, fluorescence microscopy, and genome sequencing as described below.

Visual inspection: after the seeds were germinated and the plants grown, plants were removed from the soil. It was observed that roots grown in the presence of the fungus were much hairier and more bifurcated than plants grown in the absence of the fungus, suggesting that the fungus became associated with the root in the experimental plants. See, for example, FIG. 6 and FIGS. 7A and 7B.

Fluorescence microscopy: since bacterial associations cannot be visually ascertained, bacterial staining was used to verify the presence of the nitrogen-fixing bacteria on the experimental plants. 21-day old roots were stained with the nucleic acid stain, Hoechst 33342, and imaged under 40× magnification using a Zeiss Axio Vert. Hoechst stain clearly identified the bacteria. FIGS. 8A-8E show that, microscopically, bacteria could be detected within roots, and FIG. 9 shows imaging of mixed inoculant sorghum roots using fluorescence microscopy. Sorghum root inoculated with Intraradices fungus and Glu bacterial culture resulted in Glu cells collecting at base of emerging lateral root (FIG. 9, left). Glu and Burk are endophytic, can colonize the base of new roots and can internally invade root cortex (FIG. 9, middle), while Azotobacter is associative, present external to the root and can exist in the soil (FIG. 9, right).

Step 4 of the methods described herein requires that the necessary nutrients are transferred from the bacteria to the fungus and, ultimately, to the plant. This was experimentally demonstrated as follows. The fungi and bacteria were co-cultured in a plate, but separated by a porous membrane. N14 or N15 was introduced into a bag covering each plate, and, after 8 days, the fungal biomass was harvested (from 4 replicates) for N15/N14 measurements using mass spec. See FIG. 10.

The presence of N15 in the fungus is a clear indication that nitrogen is being transferred from the bacterial species to the fungus. Thus, these experiments demonstrated efficient transfer of N15 from the air to the fungal species via a nitrogen fixing bacteria.

Significantly, nutrient transfer was observed between bacteria and fungi when the root and the fungi were separated from the bacteria. Bacteria grown on media with no sugar was able to grow and survive because of nutrient transfer from the fungus on the other side of the dish. In return, there was nitrogen transferred from the bacteria to the fungus. The bacteria converts nitrogen from the air into NH3, which is then transferred to the fungus. While the media that was used to grow the fungus contained no active nitrogen source, bacterial-fixed nitrogen was present in the fungus. The exchange of sugar for nitrogen between the bacteria and fungus is the foundation for the symbiotic relationship between them. See FIG. 11.

To examine the types of microbial organisms present, genomic sequencing was performed on the microorganisms collected from roots. Plant roots were dipped gently in phosphate buffered saline after imaging analysis, brushed onto solid media and incubated for 3 days before DNA was isolated and sequenced. FIG. 12 shows the identification of bacterial isolates from fungus- and bacteria-inoculated sorghum plant roots based on 16S sequencing. Gluconacetobacter and Burkholderia, were found in the mixed fungus-bacteria samples. Comparison of the bacterial strains present before coating of the seeds (top) and after growth of the plants (bottom) revealed that the changes were not due to mutation.

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A method of delivering nutrients to a plant, comprising:
contacting a plurality of plant seeds with selected bacteria and a selected mycorrhizal fungus;
planting the plant seeds; and
allowing plants to grow from the plant seeds, wherein precursors produced by the bacteria are provided to the plants via the fungus, and the plants utilize the precursors.

2. The method of claim 1, wherein the plant seeds comprise C₃ plant seeds, C₄ plant seeds, or both.

3. The method of claim 1, wherein the plant seeds are from a cereal plant.

4. The method of claim 1, wherein the selected bacteria comprise a single strain.

5. The method of claim 1, wherein the selected bacteria comprise a plurality of strains.

6. The method of claim 1, wherein the selected bacteria is Azospirillum brasilense, Azospirillum lipoferum, Azotobacter, Burkholderia Unamae, Gluconacetobacter diazotrophicus, Herbaspirillum seropedicae, Paenibacillus brasilensis, or Paenibacillus durus.

7. The method of claim 1, wherein the selected mycorrhizal fungi is Glomus intraradices or Rhizophagus irregularis.

8. The method of claim 1, further comprising transfecting the fungus with the bacteria.

9. The method of claim 8, further comprising contacting a peptide with the bacteria to facilitate the transfecting.

10. The method of claim 1, wherein contacting the plurality of plant seeds with the selected bacteria and the selected mycorrhizal fungus comprises coating each seed in the multiplicity of seeds with a composition comprising the selected bacteria to yield coated seeds, and contacting the coated seeds with soil comprising the selected mycorrhizal fungus.

11. The method of claim 10, wherein coating each seed occurs before or after germination of the seed.

12. The method of claim 1, wherein contacting the plurality of plant seeds with the selected bacteria and the selected mycorrhizal fungus comprises planting the plurality of seeds in soil, and providing spores of the selected mycorrhizal fungus to the soil, wherein the spores comprise the selected bacteria.

13. The method of claim 1, wherein contacting the plurality of plant seeds with the selected bacteria and the selected mycorrhizal fungus comprises injecting the selected bacteria and the selected mycorrhizal fungus into soil containing or configured to contain the plurality of plant seeds.

14. A modified seed, comprising:
a plant seed coated in a selected bacterial strain, wherein a plant grown from the coated seed, when germinated and/or grown in the presence of a selected mycorrhizal fungus, is enriched in nutrients compared to a plant grown from a seed not coated with the selected bacterial strain and not germinated and/or grown in the presence of the selected mycorrhizal fungus.

15. The modified seed of claim 14, wherein the plant seed comprises a C₃ plant seed, a C₄ plant seed, or a cereal plant.

16. The modified seed of claim 14, wherein the selected bacterial strain is Azospirillum brasilense, Azospirillum lipoferum, Azotobacter, Burkholderia Unamae, Gluconacetobacter diazotrophicus, Herbaspirillum seropedicae, Paenibacillus brasilensis, or Paenibacillus durus.

17. The modified seed of claim 14, wherein the selected mycorrhizal fungus is Glomus intraradices or Rhizophagus irregularis.

18. A modified soil mixture, comprising:
soil provided with a selected bacteria-fungus mixture comprising a selected bacteria strain and a selected mycorrhizal fungus, wherein the bacteria-fungus mixture causes a plant grown in the modified soil mixture to synthesize compounds from precursors produced by the bacteria strain that are provided to the plant by the selected mycorrhizal fungus.

19. The modified soil mixture of claim 18, wherein the selected bacterial strain is Azospirillum brasilense, Azospirillum lipoferum, Azotobacter, Burkholderia Unamae, Gluconacetobacter diazotrophicus, Herbaspirillum seropedicae, Paenibacillus brasilensis, or Paenibacillus durus.

20. The modified soil mixture of claim 18, wherein the selected mycorrhizal fungus is Glomus intraradices or Rhizophagus irregularis.
